(19) United States
(12) Patent Application Publication
(10) Pub. No.: US 2014/0128824 A1
(43) Pub. Date: May 8, 2014

(54) Connecting device

(71) Applicant: Paul Hartmann AG, Heidenheim (DE)

(72) Inventors: Pierre Croizat, Herbrechtingen (DE); Axel Eckstein, Heidenheim (DE); Juergen Hofstetter, Heidenheim (DE); Ulrich Fink, Heidenheim (DE)

(73) Assignee: Paul Hartmann AG, Heidenheim (DE)

(21) Appl. No.: 14/151,834

(22) Filed: Jan. 10, 2014

Related U.S. Application Data
(63) Continuation of application No. 12/894,170, filed on Sep. 30, 2010.

(30) Foreign Application Priority Data
Jan. 26, 2010 (DE) 10 2010 006 272.3

(51) Int. Cl. A61M 1/00

(52) U.S. Cl. CPC A61M 1/0088
USPC 604/319

(57) ABSTRACT

A connecting device (2) for vacuum treatment of wounds has a conduit element (4) which can be evacuated and a carrier element (6), wherein the connecting device can be disposed onto a vacuum dressing (13) which extends over the wound and tightly seals it from the atmosphere. The conduit element (4) communicates with the wound space through at least one opening (16) in a wall (18) of the conduit element (4) facing the vacuum dressing and through at least one opening in the vacuum dressing (13). The conduit element (4) is flexible and flat and the conduit element (4) and the carrier element (6) are formed in one piece in that, starting from a central longitudinal section (10) which forms a longitudinal channel (12), the conduit element (4) merges on both sides into lateral wing sections (8). The flat conduit element (4) with longitudinal section (10) and lateral wing sections (8) can be widely applied onto the vacuum dressing (13).

This application is a continuation of Ser. No. 12/894,170 filed Sep. 30, 2010 and also claims Paris convention priority from DE 10 2010 006 272.3 filed Jan. 26, 2010, the entire disclosures of which are hereby incorporated by reference.

### BACKGROUND OF THE INVENTION

The invention concerns a connecting device for use with vacuum treatment of wounds, comprising a conduit means which can be evacuated, and a carrier means, wherein the connecting device can be disposed onto a vacuum dressing which extends over the wound and tightly seals it from the atmosphere, wherein the conduit means communicates with the wound space through at least one opening in the wall of the conduit means facing the vacuum dressing and through at least one opening in the vacuum dressing.

The treatment of wounds with vacuum, in particular deep wounds the healing process of which is a priori problematic, has recently become more and more important. Vacuum treatment thereby means that an area of a body or wound, which is, in principle, exposed to the surrounding atmosphere, is sealed from the surroundings, i.e. the atmosphere in which we live and breathe, in a pressure-tight or vacuum-tight fashion through means which are described in detail below, wherein a lower pressure than atmospheric pressure, consequently a vacuum compared to atmosphere, can be created and permanently maintained within the sealed wound area in a fashion which is also explained below. The vacuum mentioned in this connection means a pressure range which is typically between 0 and 500 mg Hg (mm mercury column) below the ambient atmospheric pressure. This has proven to support wound healing. A vacuum dressing is provided for vacuum-tight sealing, which may comprise e.g. a pressure-tight or vacuum-tight film layer which is typically applied to a sound body area surrounding the wound to thereby tightly seal it. For introducing and maintaining a vacuum in the wound space on the basis of a means that generates vacuum, i.e. a vacuum pump in the broadest sense, the present systems for vacuum therapy of wounds may utilize conduit means which can be evacuated and which cooperate with the vacuum dressing by means of a connecting device in order to create and maintain vacuum in the wound space. A connecting device of the above-mentioned type is disclosed e.g. in WO 2006/052338 A2. It utilizes tubular hoses for “transporting” the vacuum to the wound.

It is the underlying purpose of the present invention to create a connecting device of the above-mentioned type which is comfortable for the patient or causes minimum pain upon load or contact, the components of which can be sealed with technically and economically acceptable expense in a user-friendly fashion.

### SUMMARY OF THE INVENTION

This object is achieved in accordance with the invention in a connecting device of the above-mentioned type in that the conduit means is designed to be flexible and flat and the conduit means and the carrier means are formed in one piece in that, starting from a central longitudinal section which forms a longitudinal channel, the conduit means merges on both sides into a lateral wing section, and the flat conduit means with longitudinal section and lateral wing sections can be widely applied onto the vacuum dressing.

The conduit means therefore does not have a tubular shape with a substantially round cross section but a flat shape, wherein the extension in width is substantially larger than its thickness. In combination with the material selected, this creates a flexible conduit means which is more comfortable for the patient when contact pressure is applied to the connecting device or the conduit means. Point loading is thereby also reduced, which otherwise would cause pain and is highly problematic, in particular, in case of fresh wounds which are sensitive to pain. The flat design also reduces the danger of becoming caught or jammed.

Due to the overall flat design of the connecting device, the pressure load is distributed onto a larger surface area of the wound dressing, which is very advantageous in view of the above-mentioned problems.

The inventive one-piece design of conduit means and carrier means further supports the flat design, i.e. an overall small and very compact height of the connecting device. Irrespective thereof, the production is advantageous, since it is no longer necessary to connect the conduit means and the carrier means in a vacuum-tight fashion and moreover in such a fashion that the torsional forces acting on the conduit means are uniformly introduced into the carrier means. The inventive design of the conduit means with wing sections which extend in one piece on both sides and form the carrier means and uniformly and widely hold and support the conduit means on the vacuum dressing creates a connecting device that can be produced in a very economical fashion. Accordingly, the conduit means comprises lateral wing sections at least in an area on the vacuum dressing side. It is also feasible to produce the conduit means to have these lateral wing sections over its overall length, in particular, to extrude it in one piece. The lateral wing sections may then e.g. be separated outside of the joining area with the vacuum dressing. Accordingly, the conduit means or conduit element can be formed or extruded simultaneously in one piece together with and from the same material as the lateral wing sections.

In the case shown as a preferred example, the lateral wing sections have a longitudinal extension in the longitudinal direction of the conduit means of 2 to 10 cm, in particular of 3 to 8 cm, and a transverse extension thereto of preferably 1 to 5 cm, in particular 2 to 4 cm.

The flexible and flat conduit means is advantageously designed in such a fashion that the width of the longitudinal section, forming the longitudinal channel, of the conduit means is at least 10 mm, in particular at least 15 mm, and moreover, in particular, at least 18 mm and, in particular, at most 30 mm and moreover, in particular, at most 25 mm.

Moreover, the extension in thickness (D) of the conduit means is advantageously at most 10 mm, in particular, at most 7 mm, in particular, at most 5 mm, in particular, at most 4 mm and moreover, in particular, at most 3 mm.

It is basically feasible for the thickness of the wing sections, i.e. their dimensions perpendicularly to the plane of extension or plane of application of the connecting device or the vacuum dressing, to substantially correspond to the thickness of the central longitudinal section, forming the longitudinal channel, of the conduit means. In contrast thereto, it has turned out to be sufficient and moreover economical in view of the amount of material that is used to design the wing sections to be more flat than the longitudinal section, forming the longitudinal channel, of the conduit means and, in particular, to have a thickness of preferably 1 mm to 3 mm, in particular of 1 mm to 2 mm. It has turned out that, due to the one-piece transition of the central longitudinal section of the conduit means into the lateral wing sections, a relatively small thickness of the wing sections is sufficient to uniformly accept torsional forces without transmitting force peaks to the wound dressing.

The elastomeric material from which the conduit means is formed preferably has a Shore A hardness of maximally 60, in particular 5 to 60, in particular 10 to 60, in particular 15 to 50, in particular 15 to 40 and moreover, in particular, 15 to 35. A Shore A hardness range of 15 to 60 is preferred. The Shore A hardness is determined in accordance with DIN 53505 of August 2000, i.e. at 23° C. on a plate-shaped flat and smooth sample body of a thickness of 6 mm as described in the standard. In accordance with a preferred embodiment of the invention, the conduit means is formed on the basis of silicon.

In one preferred embodiment of the invention, the conduit means and its wing sections are designed to be flat such that in the area of application to the vacuum dressing it can be widely connected to the vacuum dressing over at least 80%, in particular at least 90% and moreover, in particular, at least 95% of its surface that perpendicularly projects onto the vacuum dressing under normal conditions of use.

Since the flat and flexible conduit means is used to supply vacuum into the wound space and, if necessary, to supply rinsing liquids or rinsing gases and to discharge wound secretion, i.e. it preferably only has a channel-forming communication function, it is proposed not to form the conduit means in a laminated fashion with several components or layers but to form it, despite its flat design, in a tubular shape, i.e. having a cross-section extending continuously in one piece from one single material in the peripheral direction.

It is also advantageous for the conduit means to have means which are formed on the interior and are designed, in particular, in one piece with the material of the conduit means for preventing collapse of the conduit means in case of vacuum load. These means for preventing collapse of the conduit means may, in particular, be provided in a conduit means of tubular shape as described above. These means for preventing collapse may e.g. be formed by ribs or projections. In a further development of this inventive idea, they advantageously extend in a continuous fashion. The conduit means can then advantageously be formed as an extrusion element. The ribs or projections are accordingly formed or extruded in one piece, simultaneously with and from the same material as the conduit means or conduit element.

It may moreover be advantageous for the conduit means to comprise several channels which are separated from each other in a pressure-tight fashion, wherein, in this case, the conduit means is also preferably formed in one piece, i.e. does not comprise a combination of several separate channel-forming means. The channels may comprise a rinsing channel which may guide a rising medium towards the end of the connecting device facing the wound, and a channel carrying a vacuum which is used to supply vacuum or is used to discharge wound secretions. Any clogging within the conduit means may thereby also be eliminated. Each channel advantageously communicates with at least one opening in the wall of the conduit means or directly with another channel.

The flat conduit means preferably extends over a certain distance in the longitudinal direction and may then merge into a normal, more torsion-resistant round hose via a transition or coupling element (not shown) which may form a plug connection or adhesive connection. The round hose is guided to a device which generates vacuum and may be designed in the form of a stationary device or a portable device that can be carried on the body of the patient. The transition or coupling element may also be designed for coupling a mufti-channel conduit means with a mufti-channel round hose. A section of 10 to 60 cm has turned out to be a useful longitudinal extension of the flat conduit means.

In view of an effective support and carrier function, it has turned out to be advantageous for the overall flat extension of the lateral wing sections in the area of application to the vacuum dressing to be at least 1.5 times the flat extension of the longitudinal section of the conduit means in this area. It has turned out to be sufficient for the above-mentioned area ratio to be at most 5, in particular at most 4, wherein a ratio of 2 to 3 has turned out to be advantageous.

The flat conduit means could e.g. have a rectangular shape as viewed in cross-section, wherein the two narrow sides could preferably be rounded. In accordance with a further embodiment of the invention, the conduit means has a trapezoidal shape as viewed in cross-section. The narrow sides then drop e.g. at an inclined angle with respect to the plane of the flat carrier means of 25° to 60°, in particular 35° to 50°, wherein the flanks of the single-sided or preferably two-sided trapezoidal shape do not necessarily have to extend in a straight line but may also be rounded.

In view of the number and size of the openings in the conduit means, it would, in principle, be feasible to only provide one single opening. However, it has turned out to be advantageous to provide several openings, in particular at least two openings per each cm of length of the conduit means in the communication area of conduit means and vacuum dressing.

The vacuum-tight connection between the flat conduit means and the upper side of the vacuum dressing facing away from the wound is of functional significance. In this connection, the use of a bonding agent in the broadest sense, in particular, an adhesive, adhesive tape or adhesive foil is proposed, wherein the flat side of the conduit means facing the vacuum dressing and, in particular, its wing sections, are coated with the bonding agent. A suitable adhesive tape is available from the company Avery Dennison (Turnhout, Belgium) under the trade name MED 21006R. It is thereby also advantageous for the above-mentioned side to be covered with a detachable release coating by the manufacturer, which is only removed directly prior to application of the connecting device. In order to prevent the openings in the flat side, facing the vacuum dressing, of the central longitudinal section which forms the longitudinal channel, from being clogged with adhesive, the bonding agent is advantageously not provided in the area of these openings. This area without bonding agent is, however, surrounded by coated areas in the peripheral direction in order to ensure reliable sealing thereof. The above-mentioned openings in the central section of the conduit means then communicate with openings or slots in the vacuum dressing to achieve vacuum communication with the wound space.

Each of the above-described features is regarded as being important to the invention by itself and in arbitrary combination with each other and with further features. Further features, details and advantages of the invention can be extracted from the following claims and the drawing and following description of a preferred embodiment of the invention.

### BRIEF DESCRIPTION OF THE DRAWING

FIG. 1 shows a perspective view of an inventive connecting device for use for the treatment of wounds with a vacuum dressing;

FIG. 2 shows a sectional view of the connecting device according to FIG. 1 (not to be taken to scale); and

FIGS. 3a to c show schematic sectional views of the conduit means in further embodiments of the connecting device (sectional plane perpendicular to the longitudinal extension).

### DESCRIPTION OF THE PREFERRED EMBODIMENT

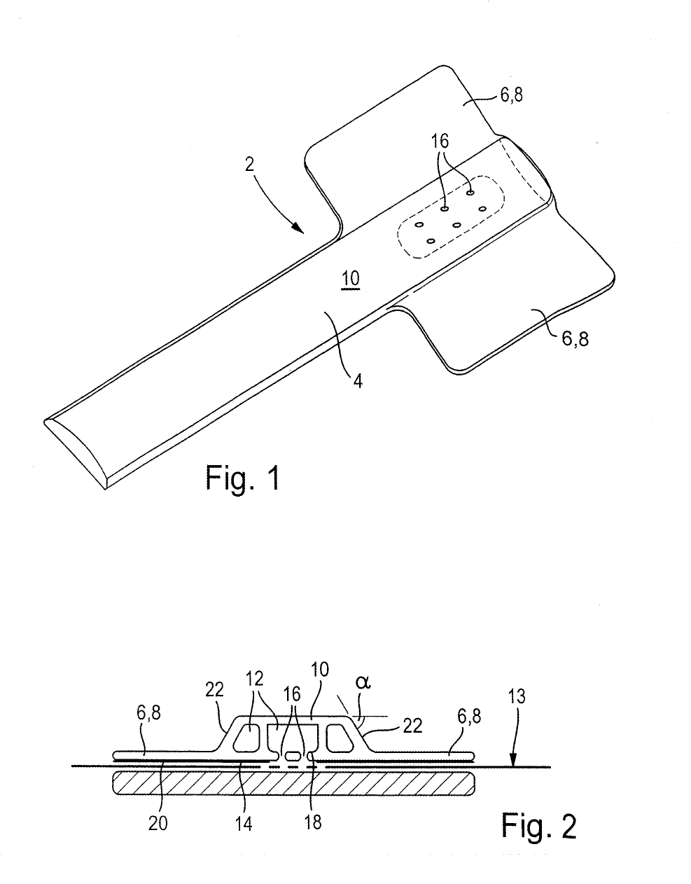

FIGS. 1 and 2 show different views of an inventive connecting device, which is designated in total with reference numeral 2, for use for vacuum treatment of wounds. The illustrated connecting device 2 is disposed, preferably in a detachable fashion, onto an upper side of a vacuum dressing facing away from the wound, which extends over a wound to be treated and seals it from the atmosphere in a vacuum-tight fashion.

The connecting device 2 comprises a flat conduit means 4 of an elastomeric flexible material with a carrier means 6 formed in one piece with the conduit means 4 and having the shape of lateral wing sections 8. The conduit means 4 thereby comprises a central longitudinal section 10 which defines in its interior at least one, in the exemplary case three, longitudinal channels 12. This central longitudinal section 10 merges in the area of its end facing the wound in one piece into the above-mentioned lateral wing sections 8, wherein these wing sections 8 in the illustrated example are even flatter, i.e. have a smaller thickness than the central longitudinal section 10, which is advantageous in accordance with the present invention but not absolutely necessary. The flat side 14 of the conduit means 4 facing the vacuum dressing 13 and comprising the lateral wing sections 8 is preferably designed to be flat, wherein a slight offset may also be provided in the area of the openings 16 in the wall 18 of the central longitudinal section 10, which is indicated with broken lines in FIG. 1.

The flat side 14 of the conduit means 4 facing the vacuum dressing and comprising lateral wing sections 8 has an adhesive coating 20, wherein the area surrounding the openings 16 may not be provided with adhesive coating. The conduit means 4 with lateral wing sections 8 may then be directly applied to the vacuum dressing 13 over the overall wide extension of the wing sections by means of this adhesive coating 20, thereby producing a vacuum-tight but preferably detachable adhesive bond. As an alternative to the adhesive coating 20, an adhesive tape of the above-mentioned type could be used.

The overall conduit means 4 with its wing sections 8 has a broad design such that it is connected to the vacuum dressing 13 with almost 100% of its surface area projected perpendicularly onto the vacuum dressing 13 in the observed contact area with the vacuum dressing 13. In an alternative fashion, which is however, less preferred than this joint between conduit means and vacuum dressing, adhesion could be realized by means of additional adhesive foils which project past the edges of the conduit means and the lateral wing sections.

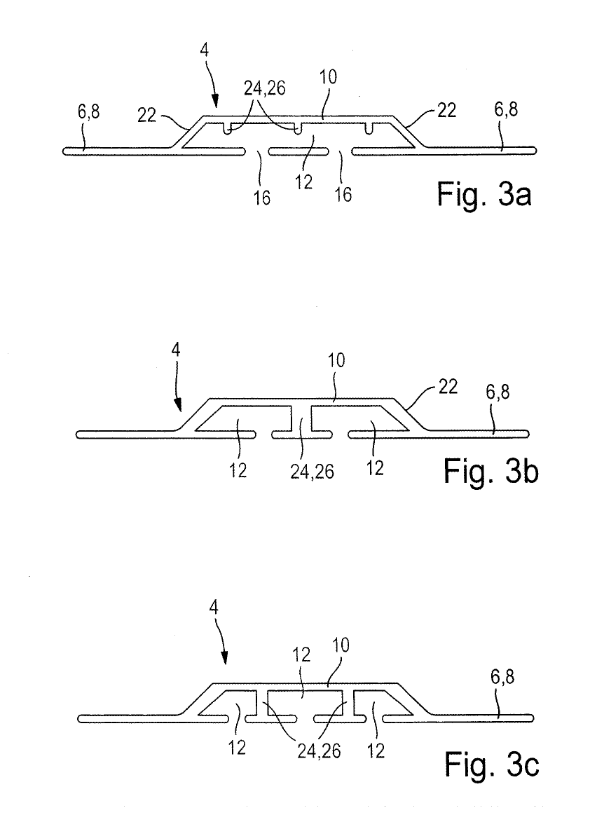

In the figures, the central longitudinal section 10 of the conduit means 4 has a trapezoidal shape and has two flanks 22 along its longitudinal extension, which drop at an inclined angle towards the carrier means and subtend an angle α of approximately 40 to 50° with the plane of extension of the conduit means 4. In the embodiments of FIGS. 1, 2, 3b and 3c, two or three channels 12 are formed in the conduit means 4, wherein one or both of the outer smaller channels may be used as rinsing line for supplying a rinsing medium. One single channel 12 which can be evacuated is formed in the conduit means 4 in accordance with FIG. 3a.

The maximum extension in thickness D of the conduit means 4 is at most 10 mm, preferably at most 7 mm, preferably at most 5 mm and moreover preferably only 2 to 4 mm.

As can be further extracted from the sectional views of FIGS. 3a to 3c, the flat conduit means 4, which is formed from a flexible material, preferably on the basis of silicon, and is moreover preferably formed in one piece, has on its inside, means 24 for preventing collapse of the conduit means 4. These means 24 are formed by ribs 26 which are continuous in the longitudinal direction and are produced in one piece with the conduit means 4. In the embodiment of the conduit means 4 in accordance with FIGS. 1, 2, 3b and 3c, these means 24 are formed by the walls (septums) between the channels 12.

We claim:

1. A device for vacuum treatment of a wound, the device comprising:
a vacuum dressing and a connecting device, said vacuum dressing and said connecting device being separate from each other prior to use on a patient, wherein said vacuum dressing is a flat sheet of material structured to extend over the wound and to attach to a sound body area surrounding the wound for vacuum-tight sealing of the wound from atmosphere;
the connecting device comprising:
a flexible and flat conduit element, said conduit element having a central longitudinal section defining at least one longitudinal channel and having a wall with at least one opening facing the vacuum dressing during use, said conduit element thereby being structured to communicate with the wound space through said at least one opening in said wall and through an opening formed in said vacuum dressing, said conduit element also having lateral wing sections formed simultaneously in one piece together with and from a same material as said central longitudinal section, said central longitudinal section and said lateral wing sections being structured for disposition onto said vacuum dressing and for evacuation thereof, wherein, starting from said central longitudinal section, said conduit element merges on both sides into said lateral wing sections, said flat conduit element with said central longitudinal and said lateral wing sections thereby being structured to spread over and cover said vacuum dressing; and
an adhesive coating disposed on a flat side of said central longitudinal and said lateral wing sections facing said vacuum dressing in order to directly apply said conduit element, with said central longitudinal and said lateral wing sections, onto said vacuum dressing.

2. The device of claim 1, wherein a width of said longitudinal section forming said longitudinal channel of said conduit element is at least 10 mm.

3. The device of claim 1, wherein a thickness of said conduit element is at most 10 mm.

4. The device of claim 1, wherein said wing sections are designed to be flatter than said longitudinal section forming said longitudinal channel of said conduit element and thereby have a thickness of 1 mm to 3 mm.

5. The device of claim 1, wherein a material from which said conduit element is formed has a Shore A hardness of maximally 60.

6. The device of claim 1, wherein said conduit element has a continuous tubular shape along a length thereof.

7. The device of claim 1, wherein said conduit element comprises an element formed on an inside or formed on an inside in one piece with material of said conduit element, for preventing collapse of said element upon vacuum load.

8. The device of claim 1, wherein said element for preventing collapse comprises ribs or projections.

9. The device of claim 8, wherein said ribs or projections extend in a longitudinal direction of said conduit element.

10. The device of claim 9, wherein said ribs or projections extend continuously in said longitudinal direction.

11. The device of claim 1, wherein said element has several channels which are separated from each other in a vacuum-tight fashion.

12. The device of claim 11, wherein the conduit means is formed in one piece.

13. The device of claim 1, wherein an overall flat extension of said lateral wing sections in an area of application to the vacuum dressing is at least 1.5 times more than a flat extension of said longitudinal section of said conduit element in the area of application to the vacuum dressing.

14. The device of claim 1, wherein, as viewed in cross-section perpendicularly to a longitudinal extension of said conduit element, said longitudinal section of said conduit element has a trapezoidal shape.

15. The device of claim 1, wherein said conduit element has at least two openings per each cm of length of said conduit element in an area of application to the vacuum dressing.

16. A device for vacuum treatment of a wound, the device comprising:
a vacuum dressing and a connecting device, said vacuum dressing and said connecting device being separate from each other prior to use on a patient, wherein said vacuum dressing is a flat sheet of material structured to extend over the wound and to attach to a sound body area surrounding the wound for vacuum-tight sealing of the wound from atmosphere;
the connecting device comprising:
a flexible and flat conduit element, said conduit element having a central longitudinal section defining at least one longitudinal channel and having a wall with at least one opening facing the vacuum dressing during use, said conduit element thereby being structured to communicate with the wound space through said at least one opening in said wall and through an opening formed in said vacuum dressing, said conduit element also having lateral wing sections formed simultaneously in one piece together with and from a same material as said central longitudinal section, said central longitudinal section and said lateral wing sections being structured for disposition onto said vacuum dressing and for evacuation thereof, wherein, starting from said central longitudinal section, said conduit element merges on both sides into said lateral wing sections, said flat conduit element with said central longitudinal and said lateral wing sections thereby being structured to spread over and cover said vacuum dressing; and
an adhesive coating disposed on a flat side of said central longitudinal and said lateral wing sections facing said vacuum dressing in order to directly apply said conduit element, with said central longitudinal and said lateral wing sections, onto said vacuum dressing, wherein said material from which said conduit element is formed has a Shore A hardness of 15 to 60, with said conduit element having a continuous tubular shape along a length thereof, said conduit element thereby comprising an element for preventing collapse of said conduit element under vacuum load, wherein said element for preventing collapse comprises ribs or projections extending continuously in a longitudinal direction of said conduit element, said ribs or projections formed in one piece with said conduit element on an inside of said conduit element, wherein said ribs or projections are formed simultaneously with said conduit element and from a same material as said conduit element.

17. A device for vacuum treatment of a wound, the device comprising:
a vacuum dressing and a connecting device, said vacuum dressing and said connecting device being separate from each other prior to use on a patient, wherein said vacuum dressing is a flat sheet of material structured to extend over the wound and to attach to a sound body area surrounding the wound for vacuum-tight sealing of the wound from atmosphere;
the connecting device comprising:
a flexible and flat conduit element, said conduit element having a central longitudinal section defining at least one longitudinal channel and having a wall with at least one opening facing the vacuum dressing during use, said conduit element thereby being structured to communicate with the wound space through said at least one opening in said wall and through an opening formed in said vacuum dressing, said conduit element also having lateral wing sections extruded simultaneously in one piece together with and from a same material as said central longitudinal section, said central longitudinal section and said lateral wing sections being structured for disposition onto said vacuum dressing and for evacuation thereof, wherein, starting from said central longitudinal section, said conduit element merges on both sides into said lateral wing sections, said flat conduit element with said central longitudinal and said lateral wing sections thereby being structured to spread over and cover said vacuum dressing; and
an adhesive coating disposed on a flat side of said central longitudinal and said lateral wing sections facing said vacuum dressing in order to directly apply said conduit element, with said central longitudinal and said lateral wing sections, onto said vacuum dressing, wherein said conduit element has a continuous tubular shape along a length thereof, said conduit element thereby comprising an element for preventing collapse of said conduit element under vacuum load, wherein said element for preventing collapse comprises ribs or projections extending continuously in a longitudinal direction of said conduit element, said ribs or projections extruded in one piece with said conduit element on an inside of said conduit element, wherein said ribs or projections are extruded simultaneously with said conduit element and from a same material as said conduit element.

18. The device of claim 1, wherein said material from which said conduit element is formed has a Shore A hardness of 15 to 60.
